(19) United States
(12) Patent Application Publication
(10) Pub. No.: US 2011/0257656 A1
(43) Pub. Date: Oct. 20, 2011

(54) TAPER SLEEVE EXTRACTOR

(75) Inventors: DAVID W. DANIELS, WARSAW, IN (US); REBECCA D. NOFTZ, WARSAW, IN (US); JOEL C. RHOADES, PIERCETON, IN (US); DANIEL N. HUFF, SIDNEY, OH (US)

(73) Assignee: DEPUY PRODUCTS, INC., WARSAW, IN (US)

(21) Appl. No.: 13/171,553

(22) Filed: Jun. 29, 2011

Related U.S. Application Data
(62) Division of application No. 11/931,495, filed on Oct. 31, 2007.

(51) Int. Cl. A61B 17/56

(52) U.S. Cl. 606/99

(57) ABSTRACT

A kit for use in orthopaedic surgery. The kit includes a plurality of heads, at least two of the plurality of heads having a different diameter. Each of the plurality of heads includes a female taper. The kit further includes a plurality of sleeves, at least two of the plurality of sleeves having a different size. Each of the plurality of sleeves has a male taper. An extractor tool is also included and has a handle and a plurality of tips, each of the plurality of tips including a flexible portion.

### CROSS-REFERENCE TO RELATED APPLICATIONS

Priority is claimed to the following application: U.S. patent application Ser. No. 11/931,495 entitled, “TAPER SLEEVE EXTRACTOR” filed on Oct. 31, 2007, by David W. Daniels, et al (Docket No. DEP5978USNP), which is herein incorporated by reference in its entirety.

### TECHNICAL FIELD OF THE INVENTION

The present invention relates generally to the field of orthopaedics, and more particularly, to an implant for use in arthroplasty.

### BACKGROUND OF THE INVENTION

Patients who suffer from the pain and immobility caused by osteoarthritis and rheumatoid arthritis have an option of joint replacement surgery. Joint replacement surgery is quite common and enables many individuals to function properly when it would not be otherwise possible to do so. Artificial joints are usually comprised of metal, ceramic and/or plastic components that are fixed to existing bone.

Such joint replacement surgery is otherwise known as joint arthroplasty. Joint arthroplasty is a well-known surgical procedure by which a diseased and/or damaged joint is replaced with a prosthetic joint. In a typical total joint arthroplasty, the ends or distal portions of the bones adjacent to the joint are resected or a portion of the distal part of the bone is removed and the artificial joint is secured thereto.

There are known to exist many designs and methods for manufacturing implantable articles, such as bone prostheses. Such bone prostheses include components of artificial joints such as elbows, hips, knees and shoulders.

Currently in total hip arthroplasty, a major critical concern is the instability of the joint. Instability is associated with dislocation. Dislocation is particularly a problem in total hip arthroplasty.

Factors related to dislocation include surgical technique, implant design, implant positioning and patient related factors. In total hip arthroplasty, implant systems address this concern by offering a series of products with a range of lateral offsets, neck offsets, head offsets and leg lengths. The combination of these four factors affects the laxity of the soft tissue. By optimizing the biomechanics, the surgeon can provide a patient a stable hip that is more resistant to dislocation.

In the case of a damaged hip joint, replacement involves resection of the proximal femur and implantation of the femoral component of an orthopaedic joint, which includes a stem part that can be received in the intramedullary canal, and a head part with a convex bearing surface. The patient's acetabulum is prepared to receive the acetabular component of the joint prosthesis, which provides a concave bearing surface to articulate with the bearing surface on the femoral component. Frequently, bone cement is used to affix the components of the prosthesis within their respective prepared bone cavities.

When the condition of the femoral bone tissue is generally good, it can be desirable to retain much of the proximal femur. Techniques have been developed in which the femoral head is fitted within a hollow resurfacing shell. The resurfacing shell has a convex outer surface that is highly polished which enables it to act against the hollow bearing surface of an acetabular component. Such techniques are referred to as Articular Surface Replacement (ASR) techniques. They have the advantage that the quantity of bone that has to be removed from the head of the bone is only small. A tool which can be used to prepare the head in this way is disclosed in International patent application no. GB03/04303.

In some ASR kits, there are a large number of femoral heads that can fit on various stem tapers with the use of sleeves. The sleeves have an inner and an outer taper. The inner taper engages with the stem and the outer taper engages with the head. The sleeves also provide various neck offsets to allow for neck length adjustments. Once the surgeon assembles the head and sleeve, it is difficult to disassemble them because of the taper. However, surgeons may sometimes need to do so to achieve better range of motion or a different neck offset. If the surgeon cannot easily remove the sleeve from the head, the surgeon must open another head and sleeve, which leads to waste.

Therefore, there is a need for an instrument that allows the surgeon to remove the sleeve from the head without ruining either the head or the sleeve.

The present invention is directed to alleviate at least some of the problems with the prior art.

### SUMMARY OF THE INVENTION

According to one embodiment of the present invention, an extractor for extracting a first component used in an orthopaedic implant from a second component used in an orthopaedic implant is provided. The extractor includes a handle and an extractor tip. The extractor tip has a proximal end and a distal end. The proximal end connects to the handle and the distal end includes a flexible portion adapted to engage the first component.

According to another embodiment of the present invention, a kit for use in orthopaedic surgery is provided. The kit includes a plurality of heads. At least two of the plurality of heads have a different diameter. Each of the plurality of heads include a female taper. The kit also includes a plurality of sleeves, at least two of the plurality of sleeves having a different size. Each of the plurality of sleeves having a male taper. The kit also includes an extractor tool. The extractor tool includes a handle and a plurality of tips. Each of the plurality of tips includes a flexible portion.

According to yet another embodiment of the present invention, a method for removing a first orthopaedic component from a second orthopaedic component is provided. The first and second orthopaedic components are joined via a taper lock. The method includes providing an extractor tool having a handle and an extractor tip that has a flexible portion. The flexible portion is inserted through a bore in the first component. The flexible portion snaps open, and a force is exerted against the handle of the extractor tool, thereby loosening the taper between the first and second components. The first component is held in place while the second component falls away, thereby removing the first component from the second component.

### BRIEF DESCRIPTION OF THE DRAWINGS

For a more complete understanding of the present invention and the advantages thereof, reference is now made to the following description taken in connection with the accompanying drawings, in which:

FIG. 1 is a perspective view of a orthopaedic implant including a head, a sleeve, and a stem according to one embodiment of the present invention;

FIG. 2 is a cross-sectional view of a head and sleeve engaged in the head according to one embodiment of the present invention;

FIG. 3 is a plan view of an extractor tool according to one embodiment of the present invention;

FIG. 4a is a cross-sectional view of the handle;

FIG. 4b is a cross-sectional view of the extractor tip of FIG. 3;

FIG. 5 is a perspective view of the handle of FIG. 3;

FIG. 6 is an exploded view of the handle of FIG. 3;

FIG. 7 is a perspective view of the extractor tip of FIG. 3;

FIG. 8 is a cross-sectional view of an extractor tip according to one embodiment of the present invention inserted into an orthopaedic implant;

FIG. 9 is a flow chart illustrating the use of the extractor tool according to one embodiment of the present invention; and

FIG. 10 is a plan view of a kit according to one embodiment of the present invention.

### DETAILED DESCRIPTION OF THE INVENTION

Embodiments of the present invention and the advantages thereof are best understood by referring to the following descriptions and drawings, wherein like numerals are used for like and corresponding parts of the drawings.

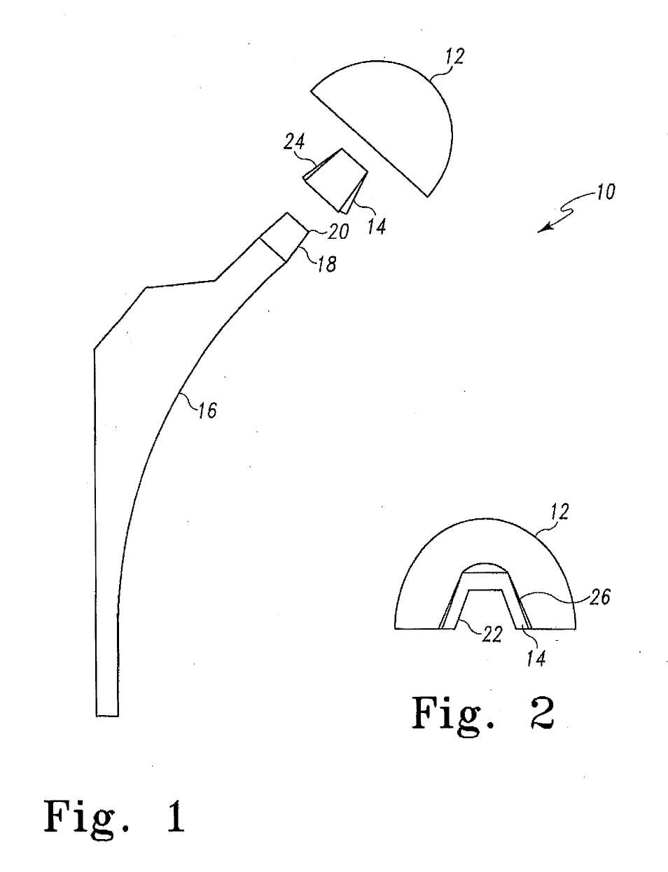

Referring now to FIG. 1, an implant 10 is illustrated. The implant 10 includes a head 12, a sleeve 14, and a stem 16. The stem 16 includes a tapered portion 18. The tapered portion 18 includes a male taper 20 that matches an internal female taper 22 on the sleeve 14 (FIG. 2). In other words, the taper angle of the internal female taper 22 of the sleeve 14 matches the taper angle of the male taper 20 of the stem 16. The sleeve 14 also includes an external male taper 24 that matches a female taper 26 of the head 12 (FIG. 2). In other words, the taper angle of the external male taper 24 of the sleeve 14 matches the taper angle of the female taper 26 of the head 12.

In assembling the head 12, sleeve 14, and stem 16, the surgeon would insert the sleeve 14 into the head 12 and lock the sleeve 14 and the head 12 via the tapers 24, 26. However, if the surgeon would need to remove the sleeve 14 from the head 12, the tapers 24, 26 are difficult, if not impossible, to disengage without the use of a tool.

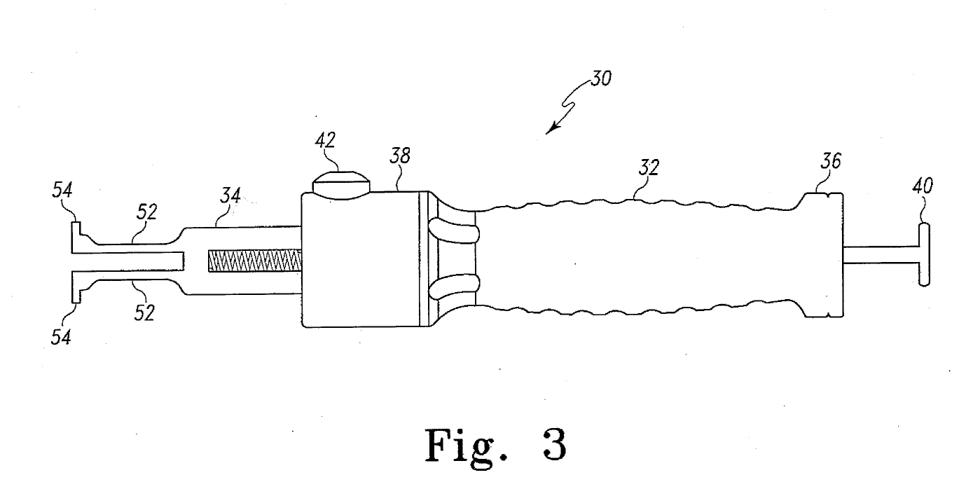

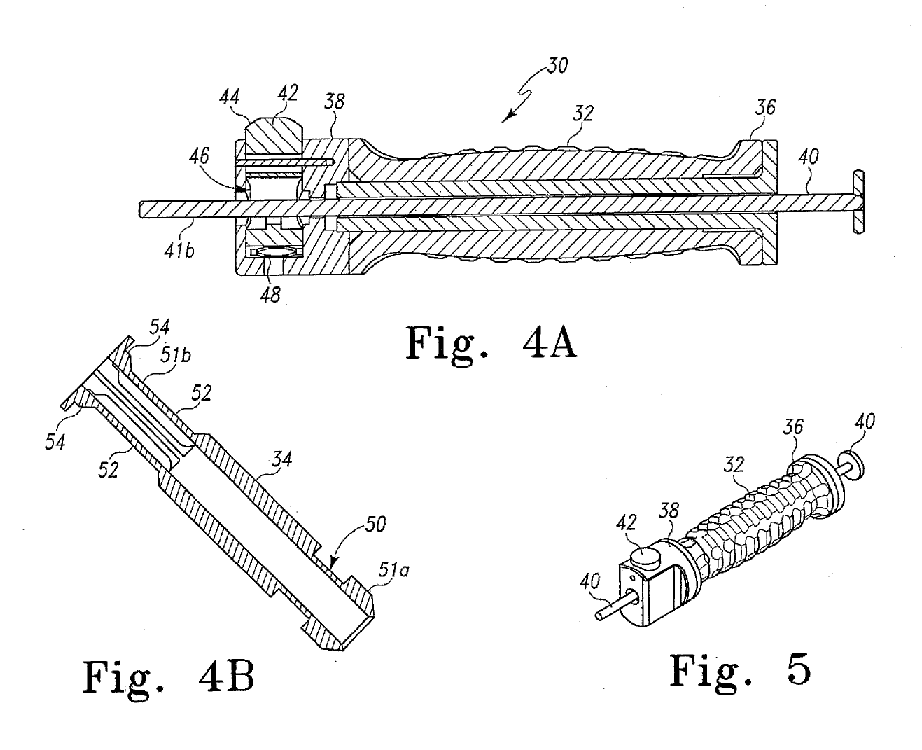

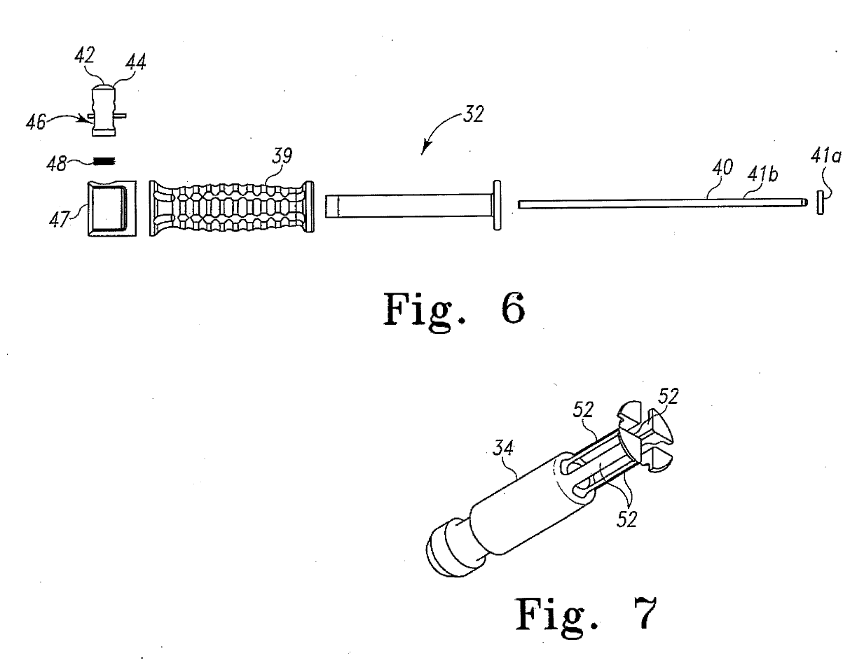

Turning now to FIG. 3, an extractor tool 30 is shown. The extractor tool 30 includes a handle 32 and an extractor tip 34. The handle 32 includes a proximal end 36 and a distal end 38. The handle 32 includes a gripping portion 39. The proximal end 36 includes an impaction mechanism 40. As shown in FIG. 6, the impaction mechanism 40 includes a plate 41a and a shaft 41b. During use of the tool, the plate 41a would be struck by the surgeon using a tool. As shown in FIGS. 4 and 5, the shaft 41b extends through the length of the handle 32. When the handle 32 is connected to the extractor tip 34, the shaft 41b will extend into the extractor tip 34, as shown in FIG. 3.

The distal end 38 is connected to the extractor tip 34. A locking mechanism 42 on the handle 32 is used to connect the extractor tip 34 to the handle 32. As shown in the cross-section view of the tool 30 in FIG. 4a the locking mechanism 42 is a spring-loaded button including a button 44, an opening 46, a button housing 47, and a spring 48. As shown in FIG. 4a, the locking mechanism 42 engages a recess 50 in the extractor tip 34. The locking mechanism 42 thus holds the extractor tip 34 in a predetermined location. If the user should wish to remove the extractor tip 34, the user presses on the button 44 and releases the extractor tip. When the button 44 is not being activated, the spring 48 exerts a force upward, locking the extractor tip 34 in place. The individual pieces of the locking mechanism 42 are shown in greater detail in the exploded view of the handle 32 in FIG. 6. Although the illustrated locking mechanism is a button utilizing a spring, other known locking mechanisms may be used. For example, the extractor tip 34 could be threaded on or could have a post/slot locking mechanism.

As shown in FIGS. 3, 4a and 4b, the extractor tip 34 includes a proximal end 51a and a distal end 51b. At the distal end 51b, the extractor tip 34 includes a flexible portion, which in this embodiment includes a plurality of spaced apart legs 52. Each leg 52 extends generally parallel to a longitudinal axis of the extractor tip 34. Distal to the legs 52, each leg 52 includes a foot 54 that extends generally tranverse to the longitudinal axis 55.

Turning now to FIG. 7, the extractor tip 34, and specifically, the legs 52 and feet 54, will be shown in greater detail. As shown in this embodiment, there are four legs 52 and four feet 54. Because of the space between each of the legs 52, the legs are somewhat flexible and can move when a force is applied against them. Although the illustrated embodiment shows four legs and four feet, it should be understood that the number of legs and feet could be varied.

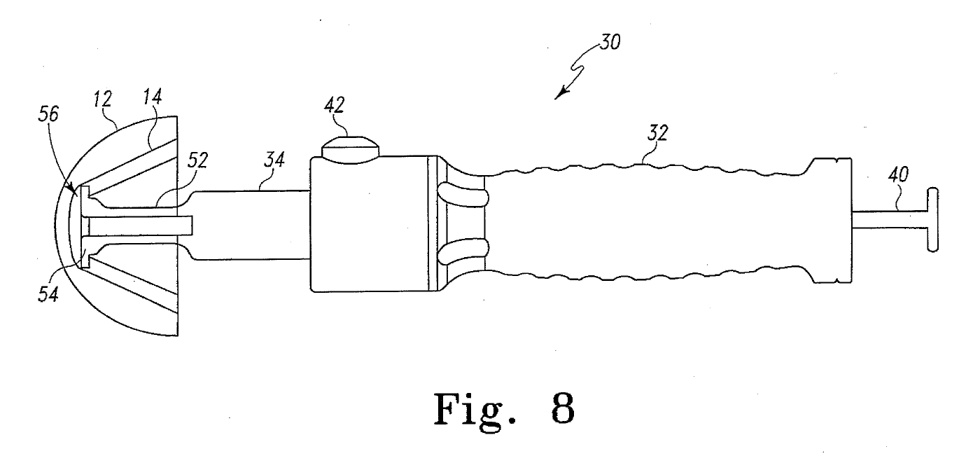

Turning now to FIG. 8, the extractor tool 30 is shown inserted into a head 12 and sleeve 14 that have been locked together. As shown, there is a small gap 56 between the top of the sleeve 14 and the inner portion of the head 12. When the extractor tip 34 is inserted into the sleeve 14, the feet 54 push into the gap 56 and then expand to grasp a top end of the sleeve 14. The impaction mechanism 40 can then be struck by a hammer or other tool, creating a vibration that travels the length of the tool 30 to the feet 54. The vibration at the feet 54 causes the tapers 24, 26 of the head 12 and sleeve 14 to be disengaged, allowing the surgeon to remove the sleeve 14 from the head 12.

The extractor tip 34 may be made of a sterilizable metal such as stainless steel. Other metals such as aluminums or radels may also be used. The gripping portion 39 of the handle 32 may be made of radel, aluminums, rubber, while the locking mechanism 42, impaction mechanism 40 and strike plate are all made of a sterilizable metal such as stainless steel. In some embodiments the extractor tool 30 may be disposable and all of the parts, except for the shaft 41b may be made of a disposable plastic such as polyethylene or radel.

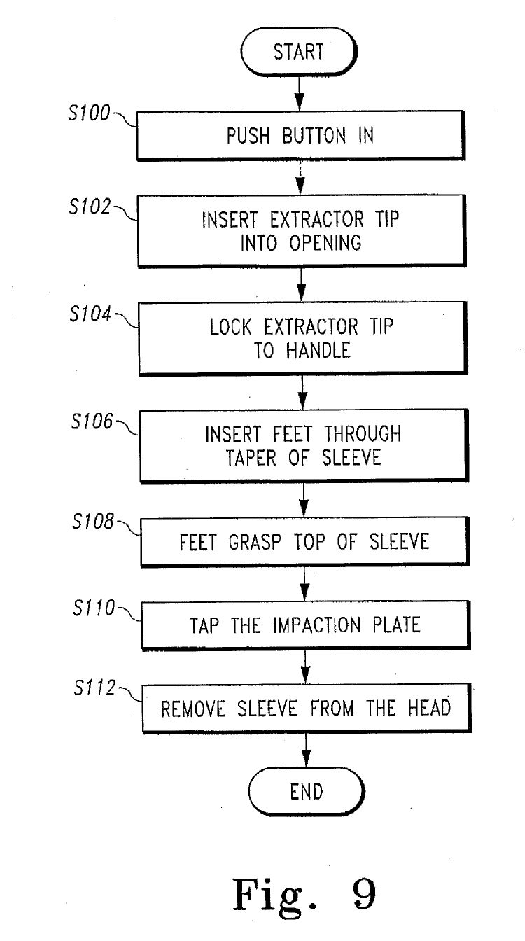

Turning now to FIG. 9, a flow chart is illustrated that describes the use of the extractor tool 30 according to one embodiment of the invention. The surgeon assembles the extractor tool 30 by pushing down the button 44 (s100) while inserting the extractor tip 34 into the opening 46 at step s102. Once the extractor tip 34 is in place, the surgeon releases the button 44, locking the extractor tip 34 to the handle 32 at step s104. The surgeon then pushes the extractor tool 30 against the head 12, thereby inserting the feet 54 of the extractor tip 34 through the internal female taper 22 of the sleeve 14 and into the gap 56 (s106). Once the feet 54 reach the gap 56 they are no longer under compression, so the feet 54 snap open into the gap 56. Once the impact force is applied, the feet 54 serve as a holding device to keep the sleeve 14 attached to the extractor tip 34 when the head falls away (step s108). Next, at step s110, the surgeon taps the impaction plate 41a with a hammer or other tool (or by hand), creating a force that travels through the handle 32 and into the extractor tool 34. The force sends a vibration through the tapers, thereby disengaging the taper between the external male taper 24 of the sleeve 14 from the female taper 26 of the head 12. The head will fall off the extractor tip, leaving the sleeve still attached at step s112.

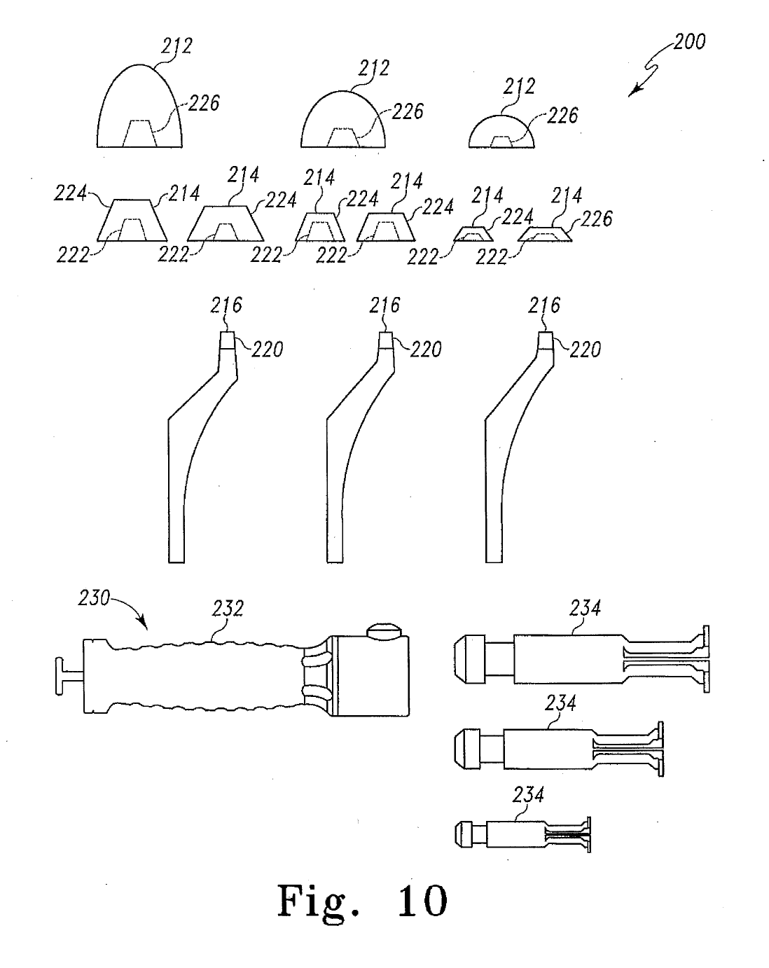

Turning now to FIG. 10, a kit 200 according to one embodiment of the present invention is shown. The kit includes a plurality of heads 212, a plurality of sleeves 214, a plurality of stems 216, and an extractor tool 230. The extractor tool 230 includes a handle 232 and a plurality of extractor tips 234. The diameters of the plurality of heads 212 are different. The heads 212 also include a female taper 226 and the taper angle of these female tapers 226 may also vary. The plurality of sleeves 214 each include an internal female taper 222 and an external male taper 224. The sleeves 214 may vary in size (e.g., length, width, etc. . . . ) as well as in taper angle. The tapers of the stems 216 may also vary.

The extractor tips 234 each include a flexible end having a plurality of legs 252, each of the legs 252 having a foot 254. The extractor tips 234 may also vary in length and in width. Because the length of the sleeves 214 may vary, the length of the legs 254 on the various extractor tips 234 may vary to fit in the selected sleeve. Also, because the taper angles of the female taper 226 of the head 212 may vary, the size of the feet 254 and/or the diameter of the extractor tip 234 may also vary. The extractor tips 234 may also vary the number of both the legs 252 and the feet 254.

In use, the surgeon would select one of the plurality of heads 212 and one of the plurality of sleeves 214 and assemble them for use. Should the surgeon need to disassemble the sleeve 214 and the head 212, the surgeon would then select an extractor tip 234 from the plurality of tips 234. The selected extractor tip would have the appropriate length and width to fit into the sleeve 214. The surgeon would then lock the selected extractor tip 234 into the handle and proceed as described in reference to FIG. 9.

Although the present invention and its advantages have been described in detail, it should be understood that various changes, substitutions, and alterations can be made therein without departing from the spirit and scope of the present invention as defined by the appended claims.

We claim:

1. A kit for use in orthopaedic surgery comprising:
a plurality of heads, at least two of the plurality of heads having a different diameter, each of the plurality of heads including a female taper;
a plurality of sleeves, at least two of the plurality of sleeves having a different size, each of the plurality of sleeves having a male taper;
an extractor tool, the extractor tool including a handle and a plurality of tips, each of the plurality of tips including a flexible portion.

2. The kit of claim 1, wherein the handle of the extractor tool includes an impaction mechanism.

3. The kit of claim 2, wherein the impaction mechanism includes a impact plate and an impact shaft, the impact shaft extending the length of the handle.

4. The kit of claim 1, wherein the handle includes a locking mechanism to lock the tip into the handle.

5. The kit of claim 4, wherein the locking mechanism is a spring-loaded button.

6. The kit of claim 1, wherein each of the plurality of extractor tips is made of a stainless steel

7. The kit of claim 1, wherein the handle includes a gripping portion and the gripping portion is made of radel.

8. A method for removing a first orthopaedic component from a second orthopaedic component, the first and second orthopaedic components being joined via a taper lock, the method comprising:
providing an extractor tool having a handle and an extractor tip, the extractor tip having a flexible portion;
inserting the flexible portion of the extractor tip through a bore in the first component;
snapping the flexible portion open;
exerting a force against the handle of the extractor tool, thereby loosening the taper between the first and second components; and
holding the first component in place while the second component falls away, thereby removing the first component from the second component.

9. The method of claim 8, wherein the flexible portion is a plurality of legs, each of the plurality of legs including a foot that extends transverse to the plurality of legs.

10. The method of claim 9, wherein the inserting the flexible portion of the extractor tip through a bore in the first component includes inserting the feet into a gap between the first component and the second component.

11. The method of claim 9, wherein the snapping open of the flexible portion of the first component is performed by the feet.

12. The method of claim 8, wherein exerting a force against the handle of the extractor tool comprises striking the handle with a tool.

13. The method of claim 8, wherein providing an extractor tool having a handle and an extractor tip, the extractor tip having a flexible portion includes inserting an extractor tip into a handle and locking the extractor tip to the handle.

14. The method of claim 13, wherein the locking the extractor tip to the handle includes using a spring-loaded button.
